(19) United States
(12) Patent Application Publication
(10) Pub. No.: US 2022/0265945 A1
(43) Pub. Date: Aug. 25, 2022

(54) NON-DISPERSIVE MULTI-CHANNEL SENSOR ASSEMBLY HAVING REFRACTIVE AND/OR DIFFRACTIVE BEAMSPLITTER

(71) Applicant: HAMILTON MEDICAL AG, Bonaduz (CH)

(72) Inventors: Christoph Schranz, Fläsch (CH); Dominik Novotni, Chur (CH); Bernd Offenbeck, Regensburg (DE)

(73) Assignee: HAMILTON MEDICAL AG, Bonaduz (CH)

(21) Appl. No.: 17/627,223

(22) PCT Filed: Jul. 15, 2020
(86) PCT No.: PCT/EP2020/069985

§ 371 Date: Jan. 14, 2022

(30) Foreign Application Priority Data
Jul. 18, 2019 (DE) 10 2019 119 502.0

(51) Int. Cl. A61M 16/00; G01N 21/3504; G01N 33/497

(52) U.S. Cl. CPC A61M 16/024; G01N 21/3504; G01N 33/497; G01N 2201/0638; G01N 2201/0634; A61M 2205/3313; A61M 2205/3303

(57) ABSTRACT

A non-dispersive multi-channel radiation sensor assembly includes a beamsplitter assembly, a first band-pass filter, which has a predefined first bandwidth and has a transmission maximum at a predefined first useful-signal wavelength, a first measurement-radiation useful-signal sensor, which is arranged downstream of the first band-pass filter in the beam path, a second band-pass filter, which has a transmission maximum at a predefined first reference-signal wavelength, a first measurement-radiation reference-signal sensor, which is arranged downstream of the second band-pass filter in the beam path. The beamsplitter assembly has a first irradiation region and a second irradiation region, in which irradiation regions the beamsplitter assembly is irradiated with measurement radiation. The irradiation regions are optically designed in such a way that the beamsplitter assembly deflects, in the first irradiation region, a first part of the measurement radiation onto the first band-pass filter and a second part of the measurement radiation onto the second band-pass filter.

The present application concerns a non-dispersive multichannel radiation sensor assembly for quantitative determination of an electromagnetic measuring radiation-absorbing component of a measuring fluid, comprising:
A beam splitter arrangement which is configured to split a beam of the measuring radiation incident on the beam splitter arrangement along a predetermined incidence axis,
A first bandpass filter with a predetermined first bandwidth and with a transmission maximum at a predetermined first useful signal wavelength reachable by a first part of the measuring radiation,
A first measuring radiation useful signal sensor arranged in the beam path behind the first bandpass filter, on which measuring radiation passing through the first bandpass filter is incident,
A second bandpass filter arranged spatially distant from the first bandpass filter, which is reachable by a second part of the measuring radiation different from the first part, where the second bandpass filter exhibits a predetermined second bandwidth and a transmission maximum at a predetermined first reference signal wavelength, where the first reference signal wavelength is different from the first useful signal wavelength, and
A first measuring radiation reference signal sensor arranged in the beam path behind the second bandpass filter and spatially distant from the first measuring radiation's useful signal sensor, on which the measuring radiation passing through the second bandpass filter is incident.

A multichannel radiation sensor assembly of the type mentioned at the beginning is known from US 2007/0241280 A1 as an infrared sensor assembly. From this publication, the use of such a sensor assembly for measuring a gas fraction, in particular CO₂, in a respiratory gas of a living patient is likewise known.

Knowledge of gas components of the respiratory gas for monitoring the vital functions of the patient and/or for monitoring the correct operation of the ventilation device is helpful and important in the artificial ventilation of live patients, be it artificial ventilation of a completely sedated or comatose patient thus unable to breathe spontaneously or be it for merely supporting ventilation of an at least periodically spontaneously breathing patient. This is sufficiently known in the relevant professional discipline. Thus for example, by detecting the CO₂ fraction in the inspiratory respiratory gas and in the expiratory respiratory gas it is possible to determine how well the metabolizing of oxygen functions in the patient. This, however, would be only one of several possible examples.

The gas sensor assembly known from US 2007/0241280 A1 uses a beam splitter which is both reflecting and transmitting, in order to split an infrared beam (IR beam) incident on the beam splitter as measuring radiation into two infrared part-beams. A first infrared part-beam is guided through a first bandpass filter onto a first sensor. This first bandpass filter exhibits a transmission maximum at the infrared (IR) absorption wavelength of CO₂ as the useful signal wavelength, and exhibits a narrow first bandwidth so that the sensor signal changes as strongly as possible as a function of the respective CO₂ content of the measuring fluid, hereinafter also referred to as “measuring gas”, traversed by the infrared beam incident on the known gas sensor assembly. The first sensor is consequently a measuring radiation and/or an infrared useful signal sensor, respectively.

After passing through a second bandpass filter and additionally through a notch filter, a second infrared part-beam is guided to a second sensor. The second bandpass filter has a transmission maximum at the infrared absorption wavelength of CO₂ as the reference signal wavelength and exhibits a greater bandwidth than the first bandpass filter. The notch filter has an extinction maximum and/or a transmission minimum respectively likewise at the infrared absorption wavelength of CO₂. The consequence is that the signal of the second sensor does not change, or changes only to a negligible extent, with the change in the CO₂ content of the measuring gas. The second sensor is consequently a measuring radiation and/or infrared reference signal sensor, respectively.

The IR reference signal sensor is needed in order to be able to assess, by comparing the signal of the IR useful signal sensor with the signal of the IR reference signal sensor, the extent of absorption of infrared light by CO₂ in the measuring gas and thereby the fraction of CO₂ in the measuring gas. This also applies to the present invention, namely regardless of whether IR radiation is used as the measuring radiation, as is preferable, or electromagnetic radiation of another wavelength.

In doing so it is helpful to derive the useful signal of the IR useful signal sensor on the one hand and the reference signal of the IR reference signal sensor on the other from one and the same incident infrared beam, in order to make sure that both the useful signal and the reference signal are exposed both qualitatively and quantitatively to essentially the same interfering factors, such that a quantitative change in the useful signal effected solely by interfering factors also effects a corresponding change in the reference signal. Thereby it is possible to prevent a change in the useful signal leading to an erroneous conclusion about a change in the gas fraction identified through the useful signal wavelength. This too applies to the present invention.

In the schematic depictions shown in US 2007/0241280 A1, the respective plane sensor detecting surfaces are inclined by 90° relative to one another. The sensor detecting surface of the IR useful signal sensor and the sensor detecting surface of the IR reference signal sensor are arranged mirror-symmetrically relative to a symmetry plane containing the virtual inclination axis.

US 2007/0241280 A1 mentions no concrete IR sensors being deployed in the known gas sensor assembly. There exist, however, IR sensors that are not only sensitive to incident infrared light, but also sensitive to mechanical stresses such as e.g. vibrations. Such mechanically sensitive IR sensors are moreover direction-dependent mechanically sensitive, i.e. a quantitatively identical mechanical stress affects one and the same IR sensor differently depending on the direction from which it acts on the IR sensor.

It is a drawback of the sensor assembly known from US 2007/0241280 A1, therefore, that a mechanical stress acting uniformly on the sensor assembly overall affects the IR useful signal sensor and the IR reference signal sensor in different ways, and consequently can cause an undesirable inaccuracy in determining the gas fraction of interest in the measuring gas.

A further drawback of the gas sensor assembly known from US 2007/0241280 A1 is the installation space required for arranging the beam splitter and the specified filters and sensors in the part-beam paths originating from it.

It is, therefore, the task of the present invention to arrange the multichannel radiation sensor assembly mentioned at the beginning both so as to be as insensitive as possible to local interference with the measuring radiation and with the smallest possible installation space and the smallest possible weight.

The present invention solves the aforementioned task by means of a non-dispersive multichannel radiation sensor assembly of the type mentioned at the beginning, in which the beam splitter arrangement is a beam splitter arrangement traversed by the measuring radiation and refracting and/or diffracting the traversing measuring radiation, where the beam splitter arrangement exhibits at least one first incidence region and at least one second incidence region spatially different from the first one. In these incidence regions measuring radiation is incident on the beam splitter arrangement, where the first and the second incidence regions are configured optically in such a way that
The beam splitter arrangement in the first incidence region
   deflects a first part of the measuring radiation incident on the first incidence region onto the first bandpass filter, and
   deflects a second part of the measuring radiation incident on the first incidence region onto the second bandpass filter,

And that
The beam splitter arrangement in the second incidence region
   deflects a first part of the measuring radiation incident on the second incidence region onto the second bandpass filter, and
   deflects a second part of the measuring radiation incident on the second incidence region onto the first bandpass filter.

Consequently, measuring radiation incident on the two incidence regions is distributed by the beam splitter arrangement to the two aforementioned bandpass filters and consequently to the measuring radiation sensors assigned to the bandpass filters: first measuring radiation useful signal sensor and first measuring radiation reference signal sensor. Interference, for instance by dirt or undesirable foreign objects in the beam path, which has only a local effect on the measuring radiation in one of the incidence regions, consequently influences the intensity of the part-measuring radiation incident on both bandpass filters and therefore on both measuring radiation sensors after passing through the beam splitter arrangement.

In principle, it can be sufficient if only one part respectively of the measuring radiation which traverses a bandpass filter falls on the measuring radiation sensor assigned to the respective bandpass filter. Preferably, the entire measuring radiation which traverses a bandpass filter falls on the measuring radiation sensor assigned to the respective bandpass filter, in order to achieve the highest possible signal level. Further preferably, there falls on a measuring radiation sensor only radiation which previously has passed through the bandpass filter assigned to the measuring radiation sensor.

Through refraction and/or diffraction of the measuring radiation traversing the beam splitter arrangement, the distribution of the measuring radiation defined above starting from different incidence regions of the beam splitter arrangement respectively to the first and the second bandpass filter and consequently to the assigned measuring radiation sensors can be realized in a very small installation space.

For the sake of simplicity, the multichannel radiation sensor assembly is also referred to below just as a ‘radiation sensor assembly’ or ‘sensor assembly’.

In accordance with a first embodiment, the first incidence region can be optically refractively active and therefore refract the measuring radiation traversing it. In order to achieve the aforementioned advantageous deflection effect, the first incidence region can exhibit at least two deflection zones which deflect the incident measuring radiation respectively in different directions. In this process, preferably at least one first deflection zone effects the deflection of the first part of the measuring radiation incident on the first incidence region onto the first bandpass filter. At least one second deflection zone preferably effects the deflection of the second part of the measuring radiation incident on the first incidence region onto the second bandpass filter. The at least one first and the at least one second deflection zone exhibit different deflection behavior with regard to the measuring radiation.

Additionally or alternatively, the second incidence region can be optically refractively active and exhibit at least two deflection zones that deflect the incident measuring radiation in different directions respectively. The second incidence region is configured as a refractively active incidence region analogous to the refractively active first incidence region described above. Consequently, at least one first deflection zone effects the deflection of the first part of the measuring radiation incident on the second incidence region. At least one second deflection zone effects the deflection of the second part of the measuring radiation incident on the second incidence region. In a refractively active second incidence region too, the at least one first deflection zone and the at least one second deflection zone differ—as in the refractively active first incidence region—in their deflection behavior with regard to the measuring radiation.

The differences in the deflection behavior between the at least one first and the at least one second deflection zone of an incidence region, regardless of whether it is the first or the second incidence region, can be due to materials differently refracting the measuring radiation of which materials the at least one first and the at least one second deflection zone are respectively made. Thus the at least one first deflection zone and the at least one second deflection zone can be made of materials with different refractive indices with respect to the measuring radiation.

Additionally or alternatively, different deflection behavior of the said deflection zones can be based on locally different interface shapes at an interface which separates the beam splitter arrangement from its environment in the region of its incidence region. Consequently, the at least one first deflection zone can exhibit a different interface shape and thus deflect measuring radiation differently than the at least one second deflection zone. Refractively active optical objects with regionally and/or zone-wise respectively different interface shapes are known in optics e.g. from Fresnel lenses. Unlike the present refractive beam splitter arrangement, in which measuring radiation incident in different zones is deflected on exiting towards different bandpass filters, Fresnel lenses exhibit a uniform focus and for reasons of desired material savings are configured with zone-wise different interface shapes in forming these lenses.

The at least one first and the at least one second deflection zone, therefore, preferably exhibit no coinciding foci and/or no uniform focal length. For the beam splitter arrangement it is rather more important to distribute the measuring radiation incident on it as evenly as possible across several bandpass filters, than to image the incident measuring radiation as exactly as possible in an image plane.

In principle it is conceivable to arrange the bandpass filters and the beam splitter arrangement with its incidence regions in such a way that every incidence region is located approximately equally distant from both bandpass filters to which it should respectively deflect parts of the incident measuring radiation. Since such a symmetry condition cannot always be reconciled with the other spatial circumstances of the sensor assembly, for the most flexible arrangement possible of the beam splitter arrangement, of the bandpass filters, and of the measuring radiation sensors, it is preferable if the at least one first and the at least one second deflection zone deflect measuring radiation incident on their incidence regions to a different extent, respectively, relative to an optical axis of the beam splitter arrangement. In the event of doubt, i.e. if due to the refractive action of the beam splitter arrangement no unambiguous optical axis can be defined, the optical axis should cross the beam splitter arrangement centrally and orthogonally to its main area of extension. If here too difficulties exist in determining an optical axis, a line parallel to the direction of incidence of the measuring radiation on the beam splitter arrangement which crosses the beam splitter arrangement as centrally as possible should be used as the optical axis.

In principle it can suffice if the first incidence region comprises only exactly one first and exactly one second deflection zone. The insensitivity to interference with the measuring radiation, however, increases with the number of first and second deflection zones, such that preferably the first incidence region comprises a plurality of first and/or of second deflection zones. For the same reasons, additionally or alternatively the second incidence region preferably comprises a plurality of first and/or of second deflection zones.

If an incidence region, be it the first and/or the second incidence region, exhibits a plurality of first and second deflection zones, this incidence region can be fabricated at the lowest possible cost by ensuring that when viewing the beam splitter arrangement in a sectional plane through the beam splitter arrangement parallel to an optical axis of the beam splitter arrangement or containing the optical axis, first deflection zones and second deflection zones of the same incidence region are arranged sequentially alternating. The first and the second deflection zones are preferably arranged in the sectional plane alternating sequentially in a direction away from the optical axis. This applies preferably to a plurality of sectional planes containing the optical axis, in particular to all sectional planes containing the optical axis. Then the beam splitter arrangement can be configured preferably rotation-symmetrically with the optical axis as the rotation symmetry axis. In the event that the aforementioned condition of a plurality of sectional planes parallel to the optical axis and to one another holds, the beam splitter arrangement can be mirror-symmetrical relative to a mirror-symmetry plane parallel to the optical axis or containing the optical axis. The said symmetrical configurations of the beam splitter arrangement reduce the susceptibility to errors of an installation of the beam splitter arrangement in a housing of the sensor assembly. In a mirror-symmetrical configuration, transposing one side of the beam splitter arrangement with the other is of no significance; in a rotation-symmetrical configuration, an angular orientation of the beam splitter arrangement about the rotation-symmetry axis is of no significance. Moreover, through a symmetrically configured beam splitter arrangement it is possible to obtain a symmetrically configured imaged spot on the exit side also, which facilitates an advantageous even distribution of measuring radiation over several bandpass filters and measuring radiation sensors.

As already described above, the first and the second deflection zone of an incidence region can differ from one another through locally different interface shapes at an interface which separates the beam splitter arrangement in the region of its incidence region from its environment. In experiments it has turned out to be advantageous here for the refracted measuring radiation proceeding from the beam splitter arrangement if an interface region exhibiting the first deflection zone and the second deflection zone exhibits a surface envelope whose distance from a tangential plane to the interface orthogonal to the optical axis, to be measured parallel to an optical axis of the beam splitter arrangement, increases with increasing distance from the optical axis of the beam splitter arrangement. Then measuring radiation incident on the beam splitter arrangement can, on the exit side of the beam splitter arrangement, be deflected advantageously evenly over a sufficiently large surface region, such that a sufficient arrangement area is available for arranging the bandpass filter and the associated measuring radiation sensors.

The refracting interface can be configured at the beam splitter arrangement on the side facing away from the bandpass filters and measuring radiation sensors and/or on the opposite side of the beam splitter arrangement facing towards the bandpass filters and measuring radiation sensors.

Preferably the surface envelope is curved convexly when viewing the beam splitter arrangement from outside, such that a refractively active section, preferably the entire refractively active section of the beam splitter arrangement, is located completely on one side of the tangential plane. The installation space needed for the beam splitter arrangement can hereby be advantageously kept small.

As a preferably rotation-symmetrically configured beam splitter arrangement, the surface envelope can exhibit a conical, a frustoconical, a convexly, or a concavely curved shape. The concavely curved shape is not preferred here, but in principle possible.

Although in principle it is possible for the refractively active beam splitter arrangement to be constructed from different, separately produced part-arrangements, it is advantageous for permanently securing the measuring radiation refracting properties of the beam splitter arrangement if at least one first deflection zone, preferably at least part of the first deflection zones, especially preferably all first deflection zones, of the first and the second incidence region at least section-wise, preferably completely, are configured as integrally connected and/or if at least one second deflection zone, preferably at least part of the second deflection zones, especially preferably all second deflection zones, of the first and the second incidence region at least section-wise, preferably completely, are configured as integrally connected.

The above non-dispersive multichannel measuring radiation sensor assembly has so far been described and further developed using the example of a two-channel measuring radiation sensor assembly. Embodiments of the sensor assembly of the present invention are not, however, limited to the two channels described above: one channel for the first useful signal and one further channel for the first reference signal. It can be a multichannel radiation sensor assembly having three-, four-, five-, six-, or eight-, or even more channels, where for each channel there is arranged one measuring radiation sensor and preferably one filter, in particular bandpass filter. Further preferably there is provided at the beam splitter arrangement for each channel its own incidence region, which on the exit side deflects measuring radiation incident on it in at least two different directions towards different measuring radiation sensors.

An especially preferred further development of the non-dispersive multichannel measuring radiation sensor assembly described above can, in order to extend its detecting scope, additionally comprise:
A third bandpass filter arranged spatially distant from the first and from the second bandpass filter, which is reachable by a third part of the measuring radiation, with a predetermined third bandwidth and with a transmission maximum at a predetermined second useful signal wavelength,
A second measuring radiation useful signal sensor arranged spatially distant from the first measuring radiation useful signal sensor and the first measuring radiation reference signal sensor, arranged in the beam path behind the third bandpass filter, on which the measuring radiation traversing the third bandpass filter is incident,
A fourth bandpass filter arranged spatially distant from the first, second, and third bandpass filter, which is reachable by a fourth part of the measuring radiation different from the first, second, and third part, where the fourth bandpass filter exhibits a predetermined fourth bandwidth and a transmission maximum at a predetermined second reference signal wavelength, where the second reference signal wavelength differs from the second useful signal wavelength,
A second measuring radiation reference signal sensor arranged in the beam path behind the fourth bandpass filter and spatially distant from the first and second measuring radiation useful signal sensor and from the first measuring radiation reference signal sensor, on which the measuring radiation traversing the fourth bandpass filter is incident,

Where the beam splitter arrangement exhibits a third incidence region spatially different from the first and from the second incidence region and a fourth incidence region spatially different from the first, second, and third incidence region, where in the third and fourth incidence regions measuring radiation is incident on the beam splitter arrangement, and where the and the fourth incidence regions are configured optically in such a way that
The beam splitter arrangement in the third incidence region
   Deflects a first part of the measuring radiation incident on the third incidence region onto the third bandpass filter, and
   Deflects a second part of the measuring radiation incident on the third incidence region onto the fourth bandpass filter,

and that
The beam splitter arrangement in the fourth incidence region
   Deflects a first part of the measuring radiation incident on the fourth incidence region onto the fourth bandpass filter, and
   Deflects a second part of the measuring radiation incident on the fourth incidence region onto the third bandpass filter.

In order to avoid unnecessary repetitions, let it be made clear: the statements made above, and likewise below, about the first incidence region, apply analogously mutatis mutandis to the third incidence region. Likewise the statements made above, and likewise below, about the second incidence region, apply analogously mutatis mutandis to the fourth incidence region. The collective term “measuring radiation sensor” consequently also covers the second measuring radiation useful signal sensor and the second measuring radiation reference signal sensor. Furthermore, the statements made above about the first bandpass filter and the first measuring radiation useful signal sensor apply analogously mutatis mutandis to the third bandpass filter and the second measuring radiation useful signal sensor. Likewise, the statements made above about the second bandpass filter and the first measuring radiation reference signal sensor apply analogously mutatis mutandis to the fourth bandpass filter and the second measuring radiation reference signal sensor.

The first useful signal wavelength is preferably a wavelength or a wavelength range of the measuring radiation, at which or in which respectively measuring radiation from a component to be detected of the measuring fluid is absorbed. The same applies preferably to the second useful signal wavelength. In this process, the second useful signal wavelength can then be quantitatively different from the first useful signal wavelength. The quantitative difference between the first and the second useful signal wavelength can be considerable, for instance when the measuring fluid exhibits two different measuring fluid components with different absorption wavelengths, of which the first absorption wavelength coincides with the first useful signal wavelength of the first measuring radiation useful signal sensor and the second absorption wavelength with a second useful signal wavelength of the second measuring radiation useful signal sensor. Then through a single multichannel radiation sensor assembly, two different measuring fluid components can be detected in one and the same measurement phase.

The useful signal wavelengths define, as described above, measuring fluid components that are detectable by the radiation sensor assembly. In principle it can be envisaged that the useful signal wavelengths differ quantitatively, although they are meant to detect the same measuring fluid component. To be able to ensure that the measuring radiation useful signal sensors can evaluate optimally the measuring radiation part incident on them with regard to the absorption information carried by it, it is advantageous if the useful signal wavelengths differ quantitatively by not more than one third of the quantitatively smaller bandwidth out of the first and third bandwidths. When detecting only one component of the measuring fluid by means of at least two measuring radiation useful signal sensors with different useful signal wavelengths respectively, preferably the useful signal wavelengths lie quantitatively on different sides of the absorption wavelength of the component. Preferably the two useful signal wavelengths differ by not more than 2%, based on the quantitatively smaller of the two wavelengths, especially preferably by not more than 1%. The first and the second useful signal wavelengths can be chosen to be quantitatively equal in order to obtain redundant signals.

When the first and the second useful signal wavelength differ quantitatively, an absorption wavelength of the component to be detected of the measuring fluid preferably exhibits a value which lies quantitatively between the first and the second useful signal wavelength, such that on a change in the quantity of the component to be detected in the measuring fluid and on an associated change in the absorption of the measuring radiation in the measuring fluid, the useful signals both of the first and of the second measuring radiation useful signal sensor change. Then an especially non-error-prone detection of one and the same component of the measuring fluid with the multichannel radiation sensor assembly can take place.

For a clean separation of the useful signal the and reference signal, preferably every wavelength out of the first and the second reference signal wavelength is different from every wavelength out of the first and the second useful signal wavelength. If only one measuring radiation useful signal sensor and one measuring radiation reference signal sensor are provided, the first reference signal wavelength and the first useful signal wavelength are different from each other. Through the described differentness it can be ensured that a useful signal is changeable as a result of changes to the measuring fluid, for instance to its composition, whereas a reference signal necessary for determining the magnitude of the change in the useful signal remains as unchanged as possible as a result of the changes in the measuring fluid and consequently essentially constant. Then the reference signal can serve as a measure for determining a change in a useful signal.

In principle it can be envisaged that each of the reference signal wavelengths is greater than each of the useful signal wavelengths or smaller than each of the useful signal wavelengths. In principle, both reference signal wavelengths can also be identical. An especially high quality of the reference signal, however, can be obtained if the two reference signal wavelengths are quantitatively different, since then undesirable random interference by a transient unexpectedly present component of the measuring fluid can only affect one of the two measuring radiation reference signal sensors. In order to provide the most robust reference signal possible, the first or the second useful signal wavelength is located quantitatively between the first and the second reference signal wavelength. Especially preferably, both useful signal wavelengths lie between the first and the second reference signal wavelength.

In order to ensure that the signal of the at least one measuring radiation useful signal sensor responds sensitively to changes in the fraction of the measuring fluid component to be detected, defined through the useful signal wavelengths, whereas the signal of the measuring radiation reference signal sensors should remain as constant as possible, it is advantageous if each bandwidth out of the second and fourth bandwidth is quantitatively smaller than each bandwidth out of the first and third bandwidth. Thus each of the second and fourth bandpass filters can exhibit a bandwidth in the two-figure nanometer range and each of the first and the third bandpass filters can exhibit a bandwidth in the three-figure nanometer range. Of the second and the fourth bandpass filters, likewise of the first and third bandpass filters, the bandpass filter with the smaller transmission maximum wavelength can exhibit the greater bandwidth. Bandwidths for the second and fourth bandpass filters preferably lie in a range from 50 to 99 nm, especially in a range from 60 to 90 nm. Bandwidths for the first and third bandpass filters preferably lie in the range from 150 to 200 nm, especially in a range from 170 to 180 nm.

The aforementioned notwithstanding, when the first reference signal wavelength provides a secure and robust reference signal, for instance because it is located far from possible or expected absorption wavelengths of respective components of the measuring fluid, just one measuring radiation reference signal sensor can suffice. The aforementioned measuring radiation reference signal sensor can then be a third measuring radiation useful signal sensor. Consequently, either a detection of the first and/or of the second measuring radiation useful signal sensor can be verified by means of the third measuring radiation useful signal sensor, or through a suitable choice of the transmission maximum of the third bandpass filter a third component of the measuring fluid can be detected by the multichannel radiation sensor assembly in one and the same measurement phase.

Instead of the aforementioned components: fourth bandpass filter and second measuring radiation useful signal sensor, the preferred embodiment of the multichannel radiation sensor assembly then exhibits:
A fourth bandpass filter arranged spatially distant from the first, second, and third bandpass filter, which is reachable by a fourth part of the measuring radiation which differs from the first, second, and third part, where the fourth bandpass filter exhibits a predetermined fourth bandwidth and a transmission maximum at a predetermined third useful signal wavelength,
A third measuring radiation useful signal sensor arranged in the beam path behind the fourth bandpass filter and spatially distant from the first and second measuring radiation useful signal sensor and from the first measuring radiation reference signal sensor, on which measuring radiation traversing the fourth bandpass filter is incident.

The two versions of the radiation sensor assembly are structurally the same. Merely the signal supplied by the combination of the fourth bandpass filter and the measuring radiation sensor assigned to the fourth bandpass filter is evaluated once as a reference signal and once as a useful signal.

The multichannel radiation sensor assembly presented here is preferably a non-dispersive CO₂— and/or a non-dispersive NOₓ—, in particular a non-dispersive NO₂ radiation sensor assembly. Preferably the measuring radiation is infrared radiation. In principle, however, the measuring radiation can be any other radiation whose wavelength spectrum contains an absorption wavelength of a component to be detected of the measuring fluid.

A preferred application of the radiation sensor assembly described here is the detection of CO₂ in inspiratory and/or expiratory respiratory gas during artificial ventilation of a patient. The first and the second useful signal wavelengths lie preferably for CO₂ detection in a range between 4.25 μm and 4.28 μm. The first reference signal wavelength can lie in the range from 3.90 μm to 4.0 μm, especially preferably at 3.95 μm, the second reference signal wavelength can lie in the range from 4.40 μm to 4.5 μm, especially preferably at 4.45 μm.

The present invention makes possible an especially installation space-saving design of the multichannel radiation sensor assembly, also due to the fact that a plurality of bandpass filters and/or a plurality of measuring radiation sensors can each be arranged in a bandpass filter arrangement plane and/or in a sensor arrangement plane respectively. Then the bandpass filters are configured as planar, where the planar bandpass filters are arranged with their extension plane parallel to the bandpass filter arrangement plane. Likewise the measuring radiation sensors preferably exhibit a sensor detection plane sensitive to measuring radiation, where the measuring radiation sensors are arranged with their respective sensor detection plane parallel to the sensor arrangement plane.

Preferably the bandpass filter arrangement plane, in which the plurality of bandpass filters are arranged, and the sensor arrangement plane, in which the plurality of measuring radiation sensors are arranged, parallel to one another. Likewise, in order to achieve the greatest possible illumination of bandpass filters and measuring radiation sensors, both the bandpass filter arrangement plane and the sensor arrangement plane are preferably oriented orthogonally to the optical axis of the beam splitter arrangement. Preferably all bandpass filters are arranged in a common bandpass filter arrangement plane. Likewise, preferably all measuring radiation sensors are arranged in a common sensor arrangement plane. In order to achieve effective space utilization, the bandpass filter and/or the measuring radiation sensors can be in a tiled arrangement in their respective arrangement plane. Preferably thereby, straight marginal sections of different bandpass filters and/or measuring radiation sensors lie in the respective arrangement planes parallel opposite or abut against one another. To be able to provide the best possible space utilization in the respective arrangement plane, the bandpass filters and/or the measuring radiation sensors can exhibit a rectangular or hexagonal base area.

Additionally or alternatively to the radiation-refracting incidence regions of the beam splitter arrangement described above, the first and/or the second incidence region and, if present, additionally or alternatively the third and/or the fourth incidence region, can have a radiation-diffracting, i.e. diffractive effect.

According to an advantageous further development of the present invention, therefore, It can be provided that the first incidence region is optically diffractively active and exhibits a diffractive structure which deflects by diffraction a measuring radiation incident on it. In this process the first incidence region deflects by diffraction measuring radiation incident on it both onto the first and onto the second bandpass filter. The diffractive structure can exhibit a diffraction grating or a comparable diffractive structure with a diffractive effect, such as e.g. a diffraction slit. Especially preferably, the diffractive structure can be configured cost-effectively as a transmission hologram.

Additionally or alternatively, the second incidence region can in the same sense be optically diffractively active and exhibit a diffractive structure which deflects by diffraction a measuring radiation incident on it. In this process the second incidence region deflects measuring radiation incident on it both onto the first and onto the second bandpass filter. For the second incidence region too, it is the case that the diffractive structure can exhibit a diffraction grating or a comparable diffractive structure with a diffractive effect, preferably for instance as a transmission hologram.

In order to be able to deflect diffractively measuring radiation incident on the beam splitter arrangement in the most different directions possible as uniformly as possible, the beam splitter arrangement can exhibit in the incident direction behind one another a plurality of diffractive structures, such that measuring radiation diffracted by a first diffractive structure is incident on a second diffractive structure and is again diffracted. Preferably the principal diffraction directions of at least two diffractive structures arranged behind one another differ from each other. Especially preferably, the principal diffraction directions of at least two diffractive structures arranged behind one another are orthogonal to one another.

Likewise, an at least section-wise diffractively active beam splitter arrangement can exhibit along its optical axis two or more refractive components arranged behind one another.

Moreover, in principle it is possible for the beam splitter arrangement to exhibit behind one another in the incident direction a diffractive structure and a structure that refracts the measuring radiation, i.e. a diffractive and a refractive structure, without the order of naming them being intended to imply an order of the arrangement.

Thus far the receiving side of the radiation sensor assembly has been described, since it is equipped especially advantageously. The described radiation sensor assembly can, taken on its own, be produced, transported, and installed as a self-contained assembly. In order to obtain a detection signal from the described radiation sensor assembly, however, a measuring radiation source is required. The latter can be present separately from the radiation sensor assembly. Preferably, however, a radiation sensor assembly that is ready for use without further procedures is provided, such that according to a further development of the present invention the multichannel radiation sensor assembly preferably exhibits at a distance from the beam splitter arrangement a measuring radiation source, which is configured to emit measuring radiation towards the beam splitter arrangement. As already described above, preferably the measuring radiation source is an infrared radiation source.

For a high radiation yield at the measuring radiation sensors and thereby for a high useful signal level, it is advantageous to be able to predict the deflection of the measuring radiation incident on the beam splitter arrangement as accurately as possible. In order to achieve this, preferably the measuring radiation source is configured to emit collimated measuring radiation. This configuration can be realized at the measuring radiation source itself, for instance by using a measuring radiation-emitting laser. Additionally or alternatively, the measuring radiation source can exhibit a collimator arrangement, which in a way that is known per se can exhibit refractive optics and/or an aperture arrangement.

Further for the simplest possible handling, the radiation sensor assembly can comprise an evaluation device which from the signals of the first measuring radiation reference signal sensor obtains reference information, which from the signals of the first measuring radiation useful signal sensor obtains useful information, and which from a comparison of the reference information and the useful information outputs information about a fraction of a measuring fluid component identified by means of the first useful signal wavelength in a measuring fluid irradiated with the measuring radiation. If the radiation sensor assembly exhibits more than one reference signal sensor and more than one useful signal sensor, their signals too are processed by the evaluation device in the aforementioned manner, depending on which further measuring radiation sensors are arranged at the radiation sensor assembly.

For the preferred deployment of the radiation sensor assembly at a ventilation device for artificial ventilation of living beings, it is preferable if the multichannel radiation sensor assembly exhibits a sensor housing with a first compartment, at or in which the beam splitter arrangement, the measuring radiation useful signal sensor, and the measuring radiation reference signal sensor are arranged, and with a second compartment located spatially distant from the first compartment, at or in which the measuring radiation source is arranged, where between the first and the second compartment there is arranged an accommodating formation for accommodating a measuring cuvette between the first and the second compartment. Then the radiation sensor assembly is spatially and structurally separate and/or separable respectively from the measuring fluid to be detected by it, such that any kind of contamination of the radiation sensor assembly by the measuring fluid in possibly clinical operation is precluded or at least minimized.

Due to the preferred use of the aforementioned multichannel radiation sensor assembly, the present invention also concerns a ventilation device for at least supportive artificial ventilation of a living patient, comprising:
A respiratory gas source,
A ventilation line arrangement, in order to conduct inspiratory respiratory gas from the respiratory gas source to a patient-side, proximal respiratory gas outlet aperture and in order to conduct expiratory respiratory gas away from a proximal respiratory gas inlet aperture,
A pressure-changing device for changing the pressure of the respiratory gas in the ventilation line arrangement,
A control device for operating the respiratory gas source and/or the pressure-changing device, and
A multichannel radiation sensor assembly according to one of the preceding Claims for detecting at least one gas component in the inspiratory and/or expiratory respiratory gas.

The respiratory gas source can be a respiratory gas reservoir, a connector formation for connecting to a building facilities respiratory gas reservoir, as often is the case in hospitals, or simply the ambient air in combination with a fan that conveys the ambient air into the ventilation line arrangement.

The present invention is elucidated in more detail hereunder by reference to the attached drawings. The drawings depict:

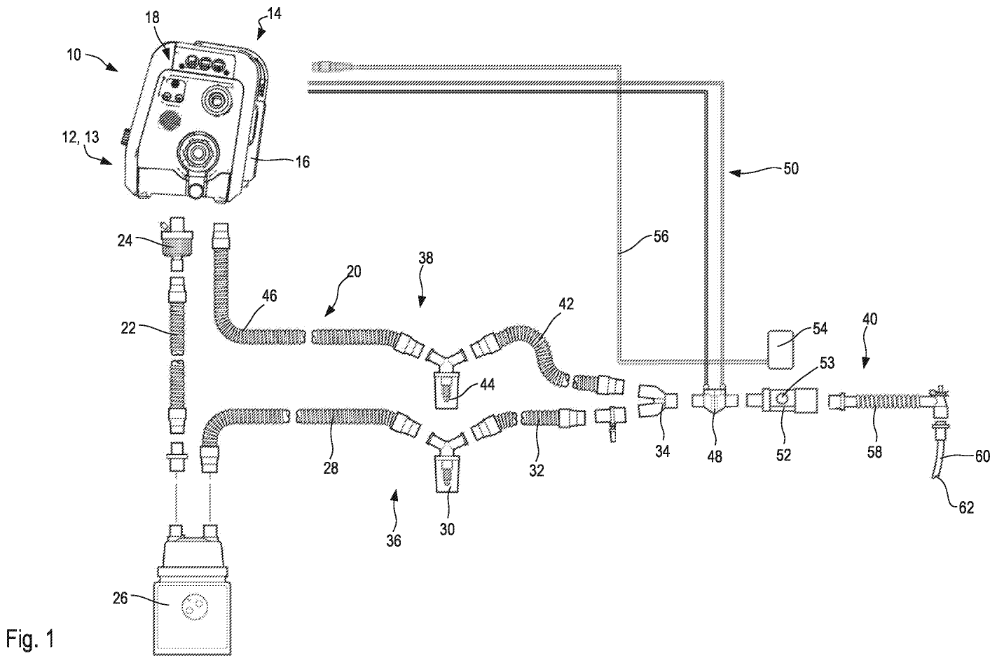

FIG. 1 A schematic exploded view of a ventilation device according to the invention,

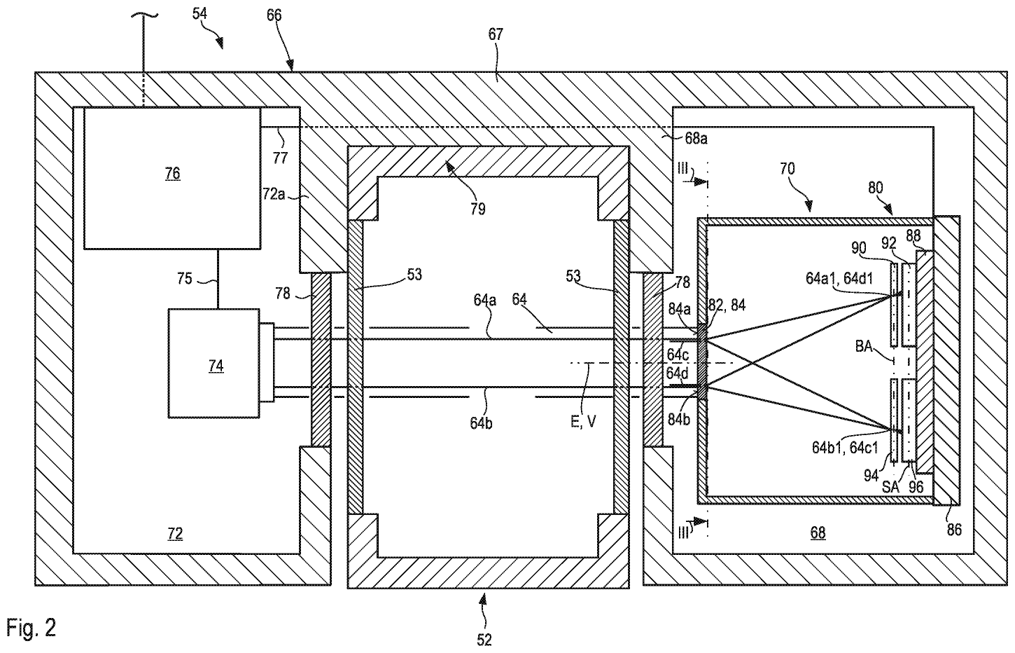

FIG. 2 A rough schematic cross-sectional view through the measuring cuvette of FIG. 1 with the invention's multichannel radiation sensor assembly of FIG. 1 accommodated in it in longitudinal section,

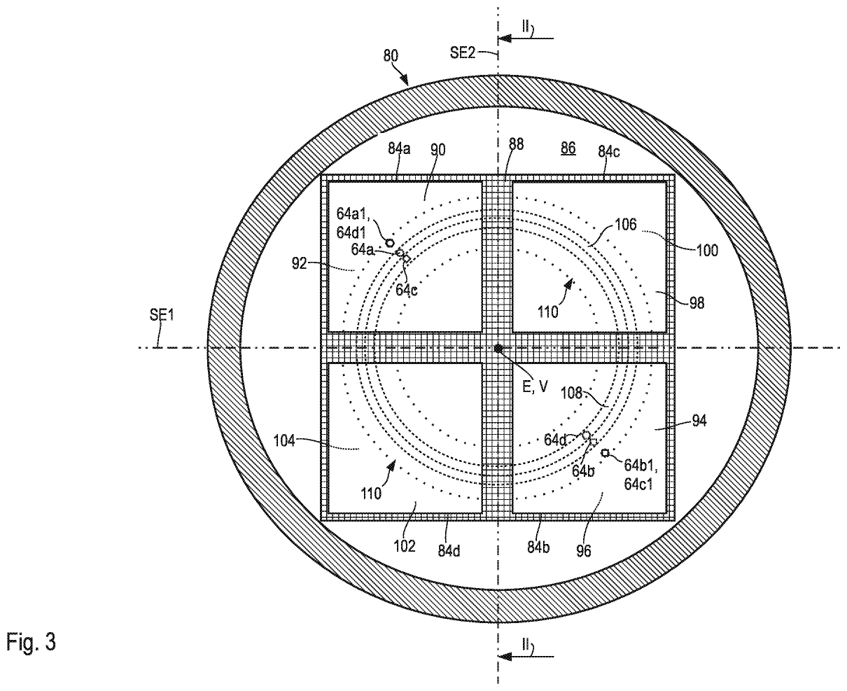

FIG. 3 A plan view of the beam splitter arrangement, showing the four bandpass filters of FIG. 2 covering their respectively assigned measuring radiation sensors when viewed along the incidence axis starting from the sectional plane III-III in FIG. 2,

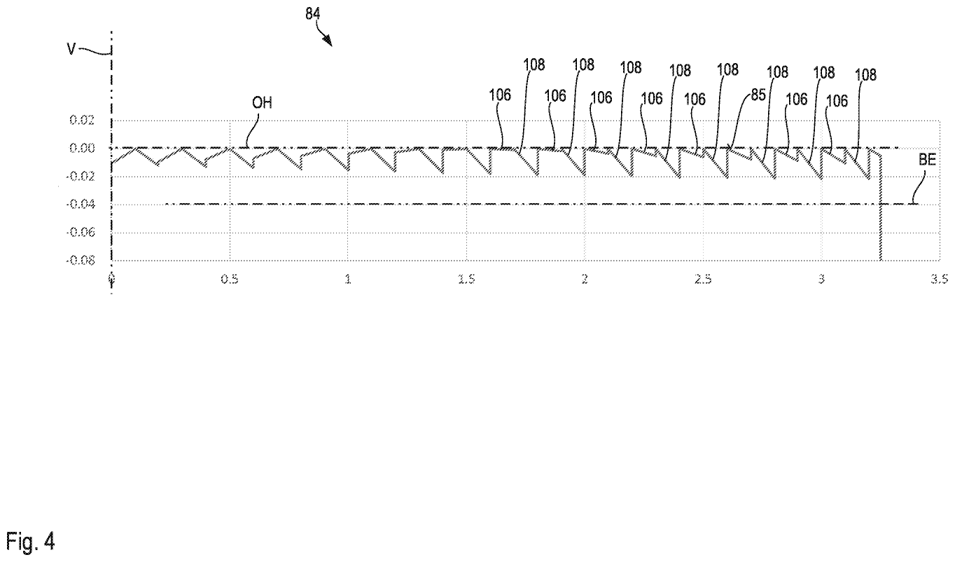

FIG. 4 A rough schematic first cross-sectional view of a possible refractively active beam splitter arrangement, and

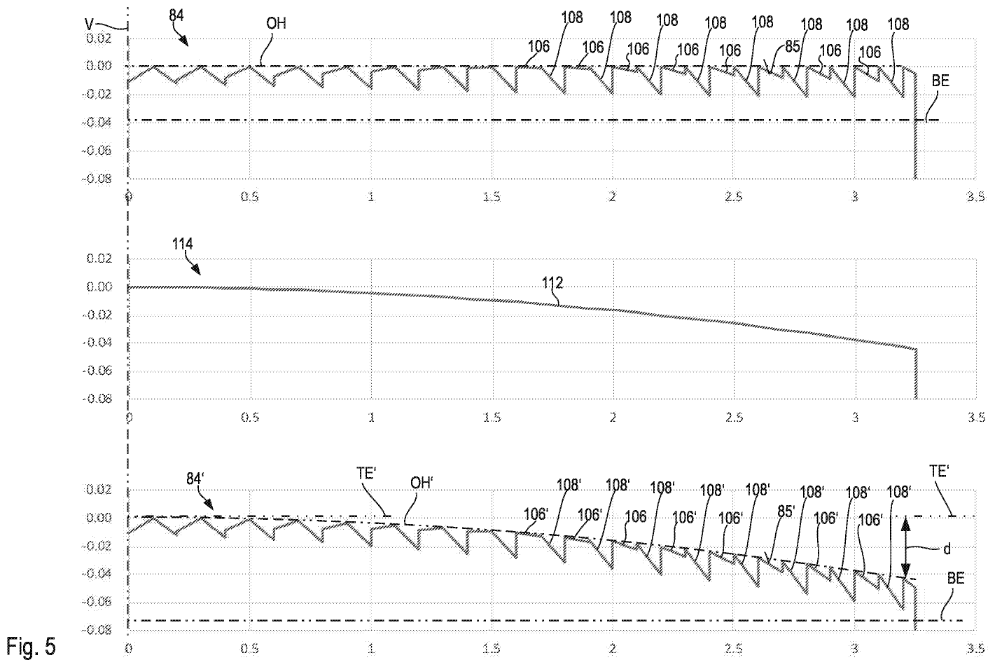

FIG. 5 A rough schematic second cross-sectional view of a preferred refractively active beam splitter arrangement with convexly curved surface envelopes.

In FIG. 1, an embodiment according to the invention of a ventilation device is denoted generally by 10. The ventilation device 10 comprises a respiratory gas source 12 in the form of a fan and a control device 14 for adjusting operational parameters of the respiratory gas source 12. The respiratory gas source 12 and the control device 14 are accommodated in the same housing 16. In this housing there are also situated valves which are known per se, such as an inspiration valve and an expiration valve. These, however, are not depicted specifically in FIG. 1.

The control device 14 of the ventilation device 10 exhibits an input/output device 18 comprising numerous switches, such as key switches and rotary switches, in order to be able to input data as required into the control device 14. The fan of the respiratory gas source 12 can be modified in its delivery rate by the control device, in order to modify the quantity of respiratory gas delivered by the respiratory gas source per unit time. The respiratory gas source 12 is therefore, in the present embodiment example, also a pressure-changing device 13 of the ventilation device.

To the respiratory gas source 12 there is connected a ventilation line arrangement 20, which in the present example comprises five flexible hoses. A first inspiratory ventilation hose 22 proceeds from a filter 24 arranged between the respiratory gas source 12 and itself to the conditioning device 26, where the respiratory gas supplied from the respiratory gas source 12 is humidified to a predetermined humidity level and if necessary provided with aerosol drugs. The filter 24 filters and cleans the ambient air supplied by the fan as the respiratory gas source 12.

A second inspiratory ventilation hose 28 leads from the conditioning device 26 to an inspiratory moisture trap 30. A third inspiratory ventilation hose 32 leads from the moisture trap 30 to a Y-connector 34, which connects the distal inspiration line 36 and the distal expiration line 38 to a combined proximal inspiratory-expiratory ventilation line 40.

From the Y-connector 34 back to the housing 16 there proceeds a first expiratory ventilation hose 42 to an expiratory moisture trap 44 and from there a second expiratory ventilation hose 46 to the housing 16, where the expiratory respiratory gas is released to the environment via a non-depicted expiration valve.

On the patient-near combined inspiratory-expiratory side of the Y-connector 34 there follows immediately after the Y-connector 34 a flow sensor 48, here: a differential pressure flow sensor 48, which detects the inspiratory and expiratory flow of respiratory gas towards the patient and away from the patient. A line arrangement 50 communicates the gas pressure prevailing on both sides of a flow obstruction in the flow sensor 48 to the control device 14, which computes from the communicated gas pressures and in particular from the difference between the gas pressures the quantity of inspiratory and expiratory respiratory gas flowing per time unit.

In the direction away from the Y-connector 34 there follows after the flow sensor 48 towards the patient a measuring cuvette 52 for non-dispersive infrared detection of a predetermined gas fraction in the respiratory gas. Consequently, the respiratory gas in the present example is the measuring fluid named in the descriptive introduction. In the present example, the measuring radiation named in the descriptive introduction is infrared radiation. The measuring fluid component to be detected in the present case is CO₂. To be determined is the fraction of CO₂ in the respiratory gas. Preferably the CO₂ fraction both in the inspiratory respiratory gas and in the expiratory respiratory gas is of interest in this process, since the change in the CO₂ fraction between inspiration and expiration is a measure of the metabolic capability of the patient's lung. In FIG. 1 it is possible to discern one of the lateral windows 53 through which infrared light can be shone into the measuring cuvette 52 and/or radiate out of it respectively, depending on the orientation of a multichannel radiation sensor assembly 54 coupled detachably with the measuring cuvette.

The sensor assembly 54 can be coupled to the measuring cuvette 52 in such a way that the sensor assembly 54 can transilluminate the measuring cuvette 52 with infrared light. From the intensity of the infrared light, more precisely from its spectral intensity, it is possible to infer, in a way that is known per se, the quantity and/or the fraction respectively of a predetermined gas in the measuring fluid and/or measuring gas respectively flowing through the measuring cuvette 52. The predetermined gas component, here: CO₂, absorbs infrared light of a defined wavelength. The intensity of the infrared light at this wavelength depends after passing through the measuring cuvette 52 essentially on the absorption of the infrared light of this wavelength by the predetermined gas component. Comparing the intensity of the infrared light of the defined wavelength with a wavelength of the infrared light that does not belong to any absorption spectrum of a gas fraction to be expected in the measuring gas, provides information about the fraction of the predetermined gas component in the measuring gas. The sensor assembly 54 is, therefore, linked via a data link 56 with the control device 14 of the ventilation device 10 and transmits the described intensity data via the data link 56 to the control device 14.

After the measuring cuvette 52 there follows in the direction towards the patient a further hose section 58, at which an endotracheal tube 60 is arranged as a ventilation interface to the patient. A proximal aperture 62 of the endotracheal tube 60 is both a respiratory gas outlet aperture through which inspiratory respiratory gas is provided to the patient through the endotracheal tube 60, and a respiratory gas inlet aperture through which expiratory respiratory gas conducted from the patient back into the endotracheal tube 60.

FIG. 2 depicts the measuring cuvette 52 in rough schematic cross-section and the multichannel radiation sensor assembly 54 coupled therein in rough schematic longitudinal section. The measuring cuvette 52 in FIG. 2 has respiratory gas flowing through it orthogonally to the drawing plane of FIG. 2. An infrared beam 64 of the sensor assembly 54 proceeds parallel to and/or in the drawing plane of FIG. 2, respectively. The drawing plane of FIG. 2 corresponds, with the exception of the position of the beam paths of the part-beams 64a, 64b, 64c, and 64d, to plane II-II in FIG. 3.

The sensor assembly 54 comprises a sensor housing 66, in whose first compartment 68 there is arranged a sensor system 70 elucidated in more detail further below, and comprises a second compartment 72, in which an infrared radiation source 74 is arranged as a measuring radiation source. Solely as an example, there is arranged in the second compartment 72 an on-board sensor control device 76, which communicates via the cables 75 and 77 with the infrared radiation source 74 and with the sensor system 70 for signal transmission and which communicates via the data link 56 with the control device 14 for signal transmission. In the present example, the control device 14 of the ventilation device 10 can function as a higher-level control device of the sensor assembly 54 and request detection values from the sensor control device 76, which thereupon actuates the infrared radiation source 74 accordingly for operation and transmits the detection signals detected by the sensor system 70 detected to the control device 14 for evaluation by the latter. The control device 14 is consequently an evaluation device of the sensor assembly 54.

The two compartments 68 and 72 are bridged by a housing bridge 67. The housing bridge 67 and the side-walls 68a and 72a adjoining it of the two compartments 68 and 72 form a clamping accommodating formation 79 into which the measuring cuvette 52 can be inserted and anchored clamped detachably. The measuring cuvette 52 and the housing 66 of the sensor assembly 54 can be separated again from one another merely by manually overcoming the clamping force. Additionally or alternatively, latching devices can be provided for latching the sensor assembly 54 and the measuring cuvette 52 to one another.

Every one of the compartments of 68 and 72 exhibits an infrared-transparent window 78 each which is traversed by the infrared beam 64 emitted by the infrared radiation source 74. Since the infrared beam 64 has to traverse the measuring cuvette 52 completely, the measuring cuvette 52 exhibits on both sides of the flow passage defined by it a window 53 each which is transparent to infrared light and which likewise is traversed by the infrared beam 64. The measuring cuvette 52 is preferably configured in the section accommodated in the sensor assembly 54 mirror-symmetrically relative to a mirror-symmetry plane orthogonal to the infrared beam 64, since the direction of the transillumination of the measuring cuvette 52 by the infrared beam 64 is of no significance. The sensor assembly 54 can, therefore, unlike the depiction in FIG. 2, also be coupled with the measuring cuvette 52 rotated by 180° about an axis orthogonal to the drawing plane of FIG. 2 and to the infrared beam 64.

The sensor system 70 exhibits its own sensor system housing 80. The sensor system housing 80 comprises a window 82 through which the infrared beam 64 can be incident on the sensor system housing 80 along an incidence axis E.

In the depicted embodiment example, the window 82 is a beam splitter arrangement 84, which deflects in different directions infrared light incident on different incidence regions. This shall be elucidated in detail hereunder. Alternatively to the configuration of the window 82 as a beam splitter arrangement 84, the window 82 can be configured as a merely transparent optically inactive window. Then a beam splitter arrangement 84 configured separately from the window 82 can be arranged behind the window 82 in the sensor system housing 80.

The sensor system housing 80 exhibits a rear wall 86 on which a substrate 88 is arranged which carries a plurality of measuring radiation and/or infrared sensors respectively 92, 96, 100 and 104 (see also FIG. 3). The substrate 88 can be configured with signal-transmitting cables in order to transmit signals between the infrared sensors 92, 96, 100, and 104 and the sensor control device 76.

In front of every infrared sensor there is situated one bandpass filter each. More precisely, a first bandpass filter 90 is situated in front of the infrared sensor 92, a second bandpass filter 94 in front of the infrared sensor 96, a third bandpass filter 98 in front of the infrared sensor 100, and a fourth bandpass filter 102 in front of the infrared sensor 104.

The first and the third bandpass filters 90 and/or 98 respectively exhibit a transmission maximum in the range of the absorption wavelengths of CO₂, approximately in a range between 4.25 μm and 4.28 μm. Their respective bandwidths lie in the range from 170 to 180 nm.

The second and the fourth bandpass filters 94 and/or 102 respectively exhibit a transmission maximum in the range outside the absorption wavelengths of CO₂, approximately in the range from 3.90 μm to 4.0 μm, and/or in the range from 4.40 μm to 4.5 μm. Their respective bandwidths lie in the range from 60 to 90 nm.

Consequently, due to the filter values of the first bandpass filter 90 set out, the infrared sensor 92 is a first measuring radiation and/or infrared useful signal sensor respectively 92 and the infrared sensor 96 is a first measuring radiation and/or infrared reference signal sensor respectively 96. Likewise, due to the filter values of the third bandpass filter 98, the infrared sensor 100 is a second measuring radiation and/or infrared useful signal sensor respectively 100. Finally, the infrared sensor 104 is a second measuring radiation and/or infrared reference signal sensor respectively 104. Given an appropriate choice of transmission maximum and bandwidth of the fourth bandpass filter 102, the infrared sensor 104 can alternatively be a third measuring radiation and/or infrared reference signal sensor respectively 104.

The infrared beam 64 is depicted in FIG. 2 in its outermost dimensions merely with a broken line. Two part-beams 64a and 64b of the infrared beam 64, to be considered in more detail in the following, are depicted with a solid line. Additionally depicted are two neighboring part-beams 64c and 64d located near the part-beams 64a and 64b of the infrared beam 64. As shown in FIG. 3 and will be elucidated in further detail, the part-beams 64a to 64d do not proceed in the drawing plane of FIG. 2, but rather partly in front of it, likewise the part-beams 64b and 64d, and partly behind, likewise the part-beams 64a and 64c. The part-beams should be understood here as local beam-regions of the infrared beam 64.

The infrared beam 64 is incident as a measuring radiation collimated infrared light along the incidence axis E, which is also the optical axis V of the beam splitter arrangement 84, on the beam splitter arrangement 84. In the course of this, the part-beams 64a and 64c are incident in a first incidence region 84a on the beam splitter arrangement 84 and the part-beams 64b and 64d are incident in a second incidence region 84b on the beam splitter arrangement 84. FIG. 3 depicts the first and second incidence regions 84a and 84b as well as a third incidence region 84c and a fourth incidence region 84d, which when viewing the beam splitter arrangement 84 along the incidence axis E of FIG. 3 coincide with the projection of the areas of the bandpass filters 90, 94, 98, and 102 along the incidence axis E onto the beam splitter arrangement 84.

The beam splitter arrangement 84 can as a refractive beam splitter arrangement 84 exhibit a plurality of first deflection zones 106 and second deflection zones 108 arranged in concentric rings about the optical axis V (see also FIG. 4), which deflect in different directions infrared radiation incident on them.

As depicted in FIG. 2 and especially in FIG. 3, the part-beam 64a is incident in the first incidence region 84a in a first deflection zone 106 on the beam splitter arrangement 84 and on the exit side is deflected onto an incidence point 64a1 on the first bandpass filter 90. The part-beam 64b is incident in the second incidence region 84b in the first deflection zone 106 on the beam splitter arrangement 84 and on the exit side is deflected onto an incidence point 64b1 on the second bandpass filter 94. The first deflection zone 106 proceeds completely and continuously around the optical axis V and consequently is a unified first deflection zone 106 for both incidence regions 84a and 84b. It deflects a part-beam which is incident parallel to the optical axis V into a virtual plane containing the optical axis V from its original path which is parallel to the optical axis.

The part-beam 64c is incident in the first incidence region 84a in the second deflection zone 108 on the beam splitter arrangement 84 and is deflected to an incidence point 64c1 on the second bandpass filter 94. The part-beam 64d is incident in the second incidence region 84b in the same second deflection zone 108 on the beam splitter arrangement 84 and is deflected onto an incidence point 64d1 on the first bandpass filter 90. The second deflection zone 108 too, deflects a part-beam which is incident parallel to the optical axis V into a virtual plane containing the optical axis V from its original path which is parallel to the optical axis. However, this deflection takes place at a different deflection angle than in the first deflection zone 106. Preferably the part-beams deflected by the second deflection zone 108, in contrast with the part-beams lying in the same plane and deflected by the first deflection zone, intersect the optical axis V.

The incidence points 64a1 and 64d1 on the first bandpass filter 90 are preferably the same incidence points or lie near one another. The same applies to the incidence points 64b1 and 64c1 on the second bandpass filter 94.

For the sake of improved clarity, out of the several first deflection zones 106 only one is shown in FIG. 3; likewise, FIG. 3 depicts only one second deflection zone 108 out of several second deflection zones 108. Along an arbitrary orthogonal line, starting from the optical axis V of the beam splitter arrangement 84, there are arranged or configured alternating sequentially first and second deflection zones 106 and/or 108 respectively.

Altogether, the infrared beam 64 incident with a circular cross-section on the beam splitter arrangement 84 is split and deflected by the the beam splitter arrangement 84 in such a way that an intensity maximum of the infrared light reaching the first to fourth bandpass filters 90, 94, 98, and 102 lies in an annular region 110 indicated by a wide-spaced dotted line. Consequently, the bandpass filters 90, 94, 98, and 102 are illuminated essentially uniformly. Due to the rotation-symmetrical configuration of the beam splitter arrangement 84 relative to the optical axis V, at least of its refractively active interface 85, with infrared radiation 64 likewise symmetrically incident relative to the optical axis V, the image produced in the region of the bandpass filters 90, 94, 98, and 102 and consequently also in the region of the measuring radiation sensors 92, 96, 100, and 104 by the beam splitter arrangement 84 is also rotation-symmetrical.

The bandpass filters 90, 94, 98, and 102, preferably configured as planar, lie in a common bandpass filter arrangement plane BA which is orthogonal to the optical axis V. Likewise, the measuring radiation sensors 92, 96, 100, and 104 which exhibit preferably planar sensor areas lie in a common sensor arrangement plane SA which is orthogonal to the optical axis V and parallel to the bandpass filter arrangement plane BA. In this way, the sensor system housing 80 and consequently essentially the sensor assembly 10 overall can be realized along the optical axis V with small dimensions.

As can be well discerned in FIG. 3, the bandpass filters 90, 94, 98, and 102 are arranged in their bandpass filter arrangement plane BA as rectangular bandpass filters in a tiled arrangement with good installation space utilization. The arrangement of the measuring radiation sensors 92, 96, 100, and 104 located in FIG. 3 behind the bandpass filters 90, 94, 98, and 102 corresponds exactly to that of the bandpass filters 90, 94, 98, and 102. Components that are adjacent to one another in the arrangement plane BA or SA respectively: bandpass filter and measuring radiation sensors, lie in the respective arrangement plane next to each other with edges parallel to one another. Due to the tiling with rectangular components, both the bandpass filters 90, 94, 98, and 102 and the measuring radiation sensors 92, 96, 100, and 104 are arranged mirror-symmetrically relative to mutually orthogonal symmetry planes SE1 and SE2 respectively. The symmetry planes SE1 and SE2 are orthogonal to the drawing plane of FIG. 3. Preferably, the intersection line of the symmetry planes SE1 and SE2 is the optical axis V. In the depicted embodiment example, moreover, the symmetry plane SE2 is the sectional plane of the depiction of FIG. 2.

FIG. 4 depicts a profile section through a possible embodiment of the beam splitter arrangement 84 in a sectional plane containing the optical axis V. The section through the refractively active interface 85 of the beam splitter arrangement 84 shows the position of the first deflection zones 106 and second deflection zones 108 alternating consecutively in the direction away from the optical axis V. The first deflection zones 106 exhibit an interface section which is less strongly inclined relative to a reference plane BE which is orthogonal to the optical axis V. By contrast, the second deflection zones 108 exhibit an interface section which is more strongly inclined relative to the reference plane BE which is orthogonal to the optical axis V. The inclination of the first and of the second deflection zones 106 and/or 108 respectively relative to the reference plane varies as a function of their distance from the optical axis V. Due to the chosen arrangement of the beam splitter arrangement 84 on the one hand and of the bandpass filters 90, 94, 98, and 102 and the measuring radiation sensors 92, 96, 100, and 104 on the other, with increasing distance from the optical axis V even the direction of inclination of the first deflection zones 106 varies, i.e. the sign of the angle at which the first deflection zones 106 are inclined to the reference plane BE. The inclination of the first deflection zones 106, therefore, increases with increasing distance from the optical axis V, since to begin with there exists a negative inclination angle in the profile section, which decreases until the sign of the inclination angle changes and the inclination angle then increases quantitatively with a positive sign with increasing distance from the optical axis V. The inclination of the second deflection zones 108 too, increases with increasing distance from the optical axis V.

For the sake of improved clarity, in FIG. 4 not all first deflection zones 106 and not all second deflection zones 108 are labelled with a reference number.

The embodiment of the beam splitter arrangement 84 of FIG. 4 exhibits a planar surface envelope OH orthogonal to the optical axis V.

In principle, a beam splitter arrangement 84 can also be deployed with a planar surface envelope OH at the radiation sensor assembly 54. However, it can then happen that the surface of the annular region 110 into which the collimated incident infrared radiation 64 is deflected by the beam splitter arrangement 84 does not illuminate optimally the surfaces of the bandpass filters 90, 94, 98, and 102 and consequently the sensor assembly 10 does indeed function, but can be further improved with regard to the achievable signal level.

FIG. 5 shows an optimized shape of the refractively active interface 85′ of an especially preferred modified beam splitter arrangement 84′. It is formed by superposition of the interface 85 of the beam splitter arrangement 84 of FIG. 4 on a convexly curved base area 112 of an optical blank 114 for producing a beam splitter arrangement 84. The result of this superposition is the refractively active interface 85′ of the modified beam splitter arrangement 84′ shown at the bottom of FIG. 5. Since there the first deflection zones 106′ and second deflection zones 108′, which are still configured in concentric rings about the optical axis V of the beam splitter arrangement 84′, are configured on a convexly curved base area 112, the refractively active interface 85′ is no longer enclosed virtually by a plane surface envelope OH but rather by a convexly curved surface envelope OH′. Through appropriate adjustment of the convex curvature of the base area 112, the radius of the annular surface 110 with the greatest intensity of the exit-side imaging of the beam splitter arrangement 84 and/or 84′ respectively can be adjusted. Consequently, the image annular surface 110 can be matched optimally through a suitable choice of the convex curvature of the base area 112 and/or of the surface envelope OH′ respectively to the actual position of the bandpass filters 90, 94, 98, and 102 and of the measuring radiation sensors 92, 96, 100, and 104.

The convex surface envelope OH′ curves at the refractively active interface 85′ of the beam splitter arrangement 84′ in every direction orthogonal to the optical axis V away from a tangential plane TE′ which is orthogonal to the optical axis V in such a way that a distance d between the surface envelope OH′ and the tangential plane TE′, measured parallel to the optical axis V, increases with increasing distance from the optical axis V. In order to avoid unnecessary material costs, preferably the entire beam splitter arrangement 84′, at least however its refractively active interface 85′, lies completely on one and the same side of the tangential plane TE′ and does not intersect the latter.

Instead of a refractively active beam splitter arrangement 84 and/or 84′ respectively, a diffractively active beam splitter arrangement can also be used, for example in the form of a transmission hologram.

1. A non-dispersive multichannel radiation sensor assembly for quantitative determination of an electromagnetic measuring radiation-absorbing component of a measuring fluid, comprising:
a beam splitter arrangement configured to split a beam of the measuring radiation incident on the beam splitter arrangement along a predetermined incidence axis,
a first bandpass filter reachable by a first part of the measuring radiation with a predetermined first bandwidth and with a transmission maximum at a predetermined first useful signal wavelength,
a first measuring radiation useful signal sensor arranged in the beam path behind the first bandpass filter on which measuring radiation traversing the first bandpass filter is incident,
a second bandpass filter arranged spatially distant from the first bandpass filter which is reachable by a second part of the measuring radiation which is different from the first part, where the second bandpass filter exhibits a predetermined second bandwidth and a transmission maximum at a predetermined first reference signal wavelength, where the first reference signal wavelength is different from the first useful signal wavelength,
a first measuring radiation reference signal sensor arranged in the beam path behind the second bandpass filter and spatially distant from the first measuring radiation useful signal sensor on which measuring radiation traversing the second bandpass filter is incident,
wherein the beam splitter arrangement is a beam splitter arrangement traversed by the measuring radiation and at least one of refracting and diffracting the traversing measuring radiation, exhibiting a first incidence region and a second incidence region different spatially from the first, in which incidence regions measuring radiation is incident on the beam splitter arrangement, where the first and the second incidence region are configured optically in such a way that
the beam splitter arrangement in the first incidence region
deflects a first part of the measuring radiation incident on the first incidence region onto the first bandpass filter, and
deflects a second part of the measuring radiation incident on the first incidence region onto the second bandpass filter,
and that
the beam splitter arrangement in the second incidence region
deflects a first part of the measuring radiation incident on the second incidence region onto the second bandpass filter, and
deflects a second part of the measuring radiation incident on the second incidence region onto the first bandpass filter.

2. The radiation sensor assembly according to claim 1, wherein at least one of the first incidence region is optically refractively active and exhibits at least two deflection zones which deflect the incident measuring radiation in different directions respectively, where a first deflection zone effects the deflection of the first part of the measuring radiation incident on the first incidence region onto the first bandpass filter and where a second deflection zone effects the deflection of the second part of the measuring radiation incident on the first incidence region onto the second bandpass filter,
and
the second incidence region is optically refractively active and exhibits at least two deflection zones which deflect the incident measuring radiation in different directions respectively, where a first deflection zone effects the deflection of the first part of the measuring radiation incident on the second incidence region and where a second deflection zone effects the deflection of the second part of the measuring radiation incident on the second incidence region,
where the first and the second deflection zone of an incidence region differ from one another through different materials with different refractive indices used in at least one of their respective configuration and through locally different interface shapes at an interface separating the beam splitter arrangement from its environment in the in the region of its incidence region.

3. The radiation sensor assembly according to claim 2, wherein the first and the second deflection zone deflect measuring radiation incident on their incidence region to a different extent, respectively, relative to an optical axis of the beam splitter arrangement.

4. The radiation sensor assembly according to claim 2,
wherein at least one of the first incidence region comprises a plurality of first and/or of second deflection zones
and
the second incidence region comprises a plurality of at least one of first and of second deflection zones.

5. The radiation sensor assembly according to claim 4, wherein along a sectional plane through the beam splitter arrangement parallel to an optical axis of the beam splitter arrangement or containing the optical axis, first deflection zones and second deflection zones of the same incidence region are arranged alternating sequentially.

6. The radiation sensor assembly according to claim 2,
wherein the first and the second deflection zone of an incidence region differ from one another through locally different interface shapes at an interface separating the beam splitter arrangement from its environment in the region of its incidence region, where an interface region exhibiting the first deflection zone and the second deflection zone exhibits a surface envelope whose distance from a tangential plane to the interface orthogonal to the optical axis, to be measured parallel to an optical axis of the beam splitter arrangement, increases with increasing distance from the optical axis of the beam splitter arrangement.

7. The radiation sensor assembly according to claim 6, wherein a refractively active section of the beam splitter arrangement is located completely on one side of the tangential plane.

8. The radiation sensor assembly according to claim 6,
wherein a refractively active section of the beam splitter arrangement is configured as mirror-symmetrical, in particular with respect to a symmetry plane containing the optical axis.

9. The radiation sensor assembly according to claim 6,
wherein a refractively active section of the beam splitter arrangement is configured rotation-symmetrically relative to the optical axis as the rotational symmetry axis.

10. The radiation sensor assembly according to claim 9, wherein the surface envelope exhibits a conical, a frustoconical, a convexly, or a concavely curved shape.

11. The radiation sensor assembly according to claim 2,
wherein at least one of at least one deflection zone of the at least one first deflection zone of the first and of the second incidence region is at least section-wise, configured as integrally connected and at least one deflection zone of the at least one second deflection zone of the first and of the second incidence region is at least section-wise, configured as integrally connected.

12. The radiation sensor assembly according to claim 1,
comprising:
a third bandpass filter arranged spatially distant from the first and from the second bandpass filter, reachable by a third part of the measuring radiation, with a predetermined third bandwidth and with a transmission maximum at a predetermined second useful signal wavelength,
a second measuring radiation useful signal sensor arranged spatially distant from the first measuring radiation useful signal sensor and from the first measuring radiation reference signal sensor, arranged in the beam path behind the third bandpass filter, on which the measuring radiation traversing the third bandpass filter is incident,
a fourth bandpass filter arranged spatially distant from the first, second, and third bandpass filter, which is reachable by a fourth part of the measuring radiation which is different from the first, second, and third part, where the fourth bandpass filter exhibits a predetermined fourth bandwidth and a transmission maximum at a predetermined second reference signal wavelength, where the second reference signal wavelength differs from the second useful signal wavelength,
a second measuring radiation reference signal sensor arranged in the beam path behind the fourth bandpass filter and spatially distant from the first and second measuring radiation useful signal sensor and from the first measuring radiation reference signal sensor on which the measuring radiation traversing the fourth bandpass filter is incident,
wherein the beam splitter arrangement exhibits a third incidence region spatially different from the first and from the second incidence region and a fourth incidence region spatially different from the first, second and third incidence region, where in the third and fourth incidence regions measuring radiation is incident on the beam splitter arrangement, and where the third and the fourth incidence region are optically configured in such a way that
the beam splitter arrangement in the third incidence region
deflects a first part of the measuring radiation incident on the third incidence region onto the third bandpass filter, and
deflects a second part of the measuring radiation incident on the third incidence region onto the fourth bandpass filter,
that
The beam splitter arrangement in the fourth incidence region
deflects a first part of the measuring radiation incident on the fourth incidence region onto the fourth bandpass filter, and
deflects a second part of the measuring radiation incident on the fourth incidence region onto the third bandpass filter.

13. The radiation sensor assembly according to claim 1,
wherein at least one of a plurality of bandpass filters and a plurality of measuring radiation sensors are each arranged in a bandpass filter arrangement plane and/or in a sensor arrangement plane respectively.

14. The radiation sensor assembly according to claim 1,
wherein at least one of the first incidence region is optically diffractively active and exhibits a diffractive structure which diffracts measuring radiation incident on it, where the first incidence region deflects measuring radiation incident on it both onto the first d onto the second bandpass filter,
and
the second incidence region is optically diffractively active and exhibits a diffractive structure which diffracts measuring radiation incident on it, where the second incidence region deflects measuring radiation incident on it both onto the first and onto the second bandpass filter.

15. The radiation sensor assembly according to claim 1,
wherein said radiation sensor assembly exhibits at a distance from the beam splitter arrangement a measuring radiation source, which is configured to emit measuring radiation towards the beam splitter arrangement.

16. The radiation sensor assembly according to claim 15,
wherein the measuring radiation source is configured to emit collimated measuring radiation.

17. The radiation sensor assembly according to claim 1,
further comprising an evaluation device which from the signals of the first measuring radiation reference signal sensor obtains reference information, which from the signals of the first measuring radiation useful signal sensor obtains useful information, and which from a comparison of the reference information and useful information outputs information about a fraction of a gas component of a measuring gas exposed to the measuring radiation identified by means of the first useful signal wavelength.

18. The radiation sensor assembly according to claim 15,
wherein the radiation sensor assembly exhibits a sensor housing with a first compartment, at or in which the beam splitter arrangement, the measuring radiation useful signal sensor, and the measuring radiation reference signal sensor are arranged, and with a second compartment located spatially distant from the first compartment, at or in which the measuring radiation source is arranged, where between the first and the second compartment there is arranged an accommodating formation for accommodating a measuring cuvette between the first and the second compartment.

19. A ventilation device for at least supportive artificial ventilation of a living patient, comprising:
a respiratory gas source,
a ventilation line arrangement, in order to conduct inspiratory respiratory gas from the respiratory gas source to a patient-side, proximal respiratory gas outlet aperture and in order to conduct expiratory respiratory gas away from a proximal respiratory gas inlet aperture,
a pressure-changing device for changing the pressure of the respiratory gas in the ventilation line arrangement,
a control device for operating at least one of the respiratory gas source and the pressure-changing device (13), and
a multichannel radiation sensor assembly according to claim 1 for detecting at least one of at least one gas component in the inspiratory and expiratory respiratory gas.

20. The radiation sensor assembly according to claim 2,
wherein at least one of at least one deflection zone of the at least one first deflection zone of the first and of the second incidence region is completely configured as integrally connected and at least one deflection zone of the at least one second deflection zone of the first and of the second incidence region is completely, configured as integrally connected.
